(19) United States
(12) Patent Application Publication
(10) Pub. No.: US 2025/0018120 A1
(43) Pub. Date: Jan. 16, 2025

(54) AUTO-INJECTOR RETAINER AND DRUG ADMINISTRATION DEVICE

(71) Applicant: TERUMO KABUSHIKI KAISHA, Tokyo (JP)

(72) Inventors: Junichi OGAWA, Kofu-shi (JP); Remi NUNOME, Chuo-shi (JP)

(73) Assignee: TERUMO KABUSHIKI KAISHA, Tokyo (JP)

(21) Appl. No.: 18/897,130

(22) Filed: Sep. 26, 2024

Related U.S. Application Data
(63) Continuation of application No. PCT/JP2022/045545, filed on Dec. 9, 2022.

(30) Foreign Application Priority Data
Mar. 28, 2022 (JP) 2022-052079

(51) Int. Cl. A61M 5/20; A61M 5/32

(52) U.S. Cl. CPC A61M 5/204; A61M 5/3295

(57) ABSTRACT

An auto-injector retainer for use in simultaneous administration by a plurality of auto-injectors each having a housing that has a cylindrical shape and houses a prefilled syringe and a gasket pushing mechanism, the auto-injector retainer including: a plurality of retaining units located in parallel, each retaining unit configured to retain a respective one of the auto-injectors. Each of the retaining units includes a casing that covers a proximal end of the housing of the respective auto-injector. Each casing includes: a distal opening into which the proximal end of the housing of the respective auto-injector is insertable, a proximal wall configured to abut on or face a proximal end face of the housing of the respective auto-injector, and a cylindrical peripheral wall configured to elastically deform when the proximal end of the housing of the respective auto-injector is housed in the casing.

### CROSS-REFERENCE TO RELATED APPLICATIONS

This is a bypass continuation of PCT Application No. PCT/JP2022/045545, filed on Dec. 9, 2022, which claims priority to Japanese Patent Application No. 2022-052079, filed on Mar. 28, 2022. The entire contents of these application are incorporated herein by reference.

### BACKGROUND

The present disclosure relates to an auto-injector retainer and a drug administration device for assisting simultaneous administration by a plurality of auto-injectors.

### BACKGROUND ART

In the related art, there is known a device (auto-injector) for automatically or semi-automatically injecting (administering) a medical liquid (drug). In such an auto-injector, for example, as disclosed in Japanese Patent Publication No. 2015-533562A (US Patent Publication No. 2015-273162A, EP Patent Publication No. 2903670B, PCT Publication No. WO2014/053451A; “Patent Literature 1”), a driving mechanism (90) for delivering a certain amount of drug is released by a predetermined operation, and the drug in a drug container (24) is ejected through a delivering member (28).

### SUMMARY

Auto-injectors are standardized to some extent depending on types and applications of medical liquids. Therefore, some patients may require a plurality of auto-injectors to administer a medical liquid according to an appropriate dose of the medical liquid.

Using a plurality of auto-injectors to administer a medical liquid increases the time and effort for the administration. To solve this problem, what is called simultaneous administration is performed to operate a plurality of auto-injectors at the same time. However, the simultaneous administration by a plurality of auto-injectors complicates the operation and is not always performed appropriately. In addition, depending on medical liquids, a problem may occur if administration sites (injection sites) of a medical liquid to be administered by a plurality of auto-injectors are not appropriate (for example, if the administration sites are too close).

An object of the present invention is to provide an auto-injector retainer and a drug administration device for assisting simultaneous administration by a plurality of auto-injectors. The auto-injector retainer and the drug administration device appropriately retain the plurality of auto-injectors in parallel without inhibiting functions of the auto-injectors.

The object is achieved by certain embodiments of an auto-injector retainer according to the disclosure.

According to one embodiment, an auto-injector retainer is capable of retaining a plurality of auto-injectors each having a housing that has a cylindrical shape and houses a prefilled syringe and a gasket pushing mechanism. The auto-injector retainer is used for assisting simultaneous administration by the plurality of auto-injectors, and includes: a plurality of retaining units capable of retaining the auto-injectors and placed in parallel. The retaining units each include a casing that covers a proximal end of the housing of each of the auto-injectors from outside to house the proximal end. The casing includes a distal opening into which the proximal end of the housing is inserted, a proximal wall abutting on or facing a proximal end face of the housing, and a cylindrical peripheral wall elastically deformed when the proximal end of the housing is housed in the casing to hold the proximal end of the housing housed in the casing by an elastic restoring force.

Furthermore, object is achieved by certain embodiments of a drug administration device according to the disclosure.

According to one embodiment, a drug administration device includes: the auto-injector retainer; and at least two of the auto-injectors.

### BRIEF DESCRIPTION OF THE DRAWINGS

FIG. 1 is a perspective view illustrating an auto-injector retainer of the present invention retaining auto-injectors.

FIG. 2 is a front view illustrating an embodiment of the auto-injector retainer of the present invention.

FIG. 3 is a plan view of the auto-injector retainer illustrated in FIG. 2.

FIG. 4 is a bottom view of the auto-injector retainer illustrated in FIG. 2.

FIG. 5 is a left side view of the auto-injector retainer illustrated in FIG. 2.

FIG. 6 is a cross-sectional view taken along line A-A of FIG. 3.

FIG. 7 is a front view illustrating an example of an auto-injector retained by the auto-injector retainer of the present invention.

FIG. 8 is a front view for describing actuation of the auto-injector illustrated in FIG. 7.

FIG. 9 is a front view for describing a state where the auto-injector retainer illustrated in FIG. 2 retains auto-injectors.

FIG. 10 is a cross-sectional view taken along line B-B in FIG. 9.

FIG. 11 is a cross-sectional view taken along line C-C in FIG. 9.

FIG. 12 is a front view for describing completion of administration of a medical liquid using the auto-injector retainer illustrated in FIG. 2.

FIG. 13 is a cross-sectional view illustrating another embodiment of the auto-injector retainer of the present invention, corresponding to FIG. 6.

FIG. 14 is a bottom view of the auto-injector retainer illustrated in FIG. 13.

FIG. 15 is a perspective view illustrating the auto-injector retainer of the present invention retaining auto-injectors.

FIG. 16 is a front view illustrating another embodiment of the auto-injector retainer of the present invention.

FIG. 17 is a plan view of the auto-injector retainer illustrated in FIG. 16.

FIG. 18 is a bottom view of the auto-injector retainer illustrated in FIG. 16.

FIG. 19 is a left side view of the auto-injector retainer illustrated in FIG. 16.

FIG. 20 is a cross-sectional view taken along line D-D in FIG. 17.

FIG. 21 is a front view illustrating another embodiment of the auto-injector retainer of the present invention.

FIG. 22 is a plan view of the auto-injector retainer illustrated in FIG. 21.

FIG. 23 is a front view illustrating another embodiment of the auto-injector retainer of the present invention.

FIG. 24 is a plan view of the auto-injector retainer illustrated in FIG. 23.

FIG. 25 is a bottom view of the auto-injector retainer illustrated in FIG. 23.

FIG. 26 is a left side view of the auto-injector retainer illustrated in FIG. 23.

FIG. 27 is a cross-sectional view taken along line E-E in FIG. 24.

FIG. 28 is a perspective view illustrating the auto-injector retainer of the present invention retaining auto-injectors.

FIG. 29 is a front view illustrating another embodiment of the auto-injector retainer of the present invention.

FIG. 30 is a plan view of the auto-injector retainer illustrated in FIG. 29.

FIG. 31 is a bottom view of the auto-injector retainer illustrated in FIG. 29.

FIG. 32 is a left side view of the auto-injector retainer illustrated in FIG. 29.

FIG. 33 is a cross-sectional view taken along line F-F in FIG. 30.

FIG. 34 is a cross-sectional view illustrating another embodiment of the auto-injector retainer of the present invention, corresponding to FIG. 10.

FIG. 35 is a front view illustrating another embodiment of the auto-injector retainer of the present invention.

### DETAILED DESCRIPTION

An auto-injector retainer of the present disclosure will be described with reference to embodiments illustrated in the drawings.

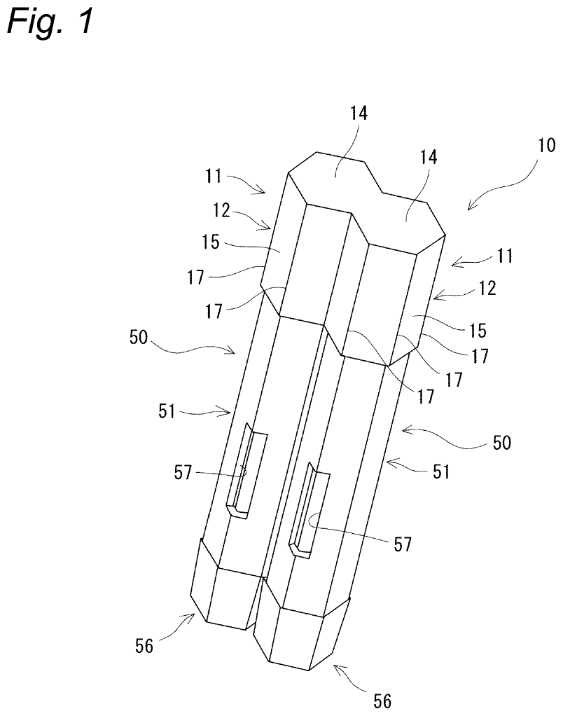

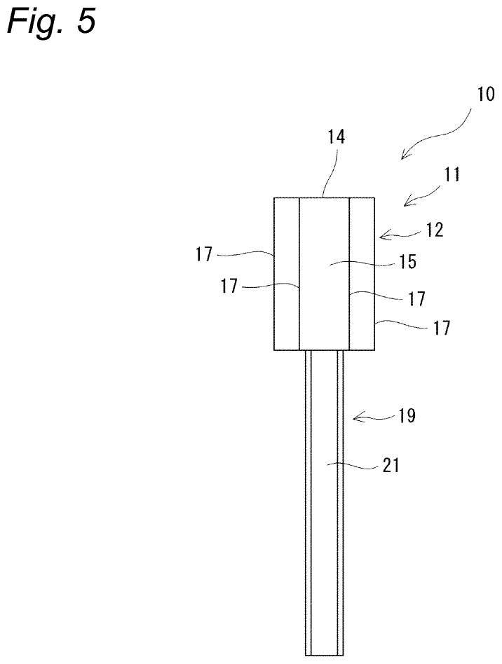

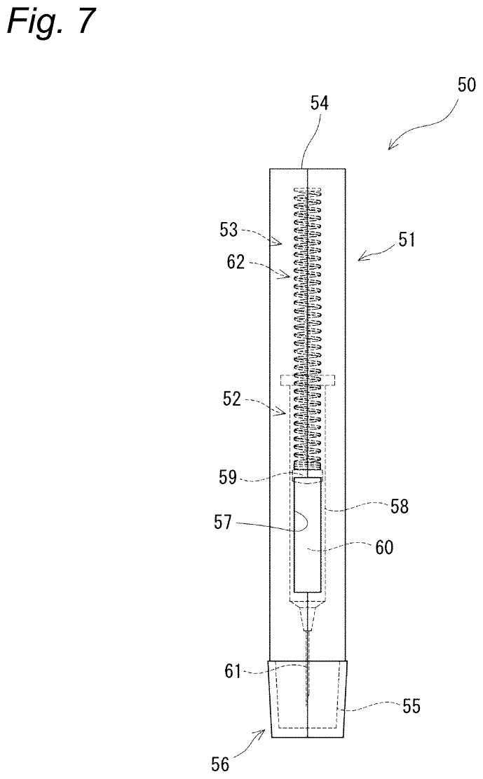

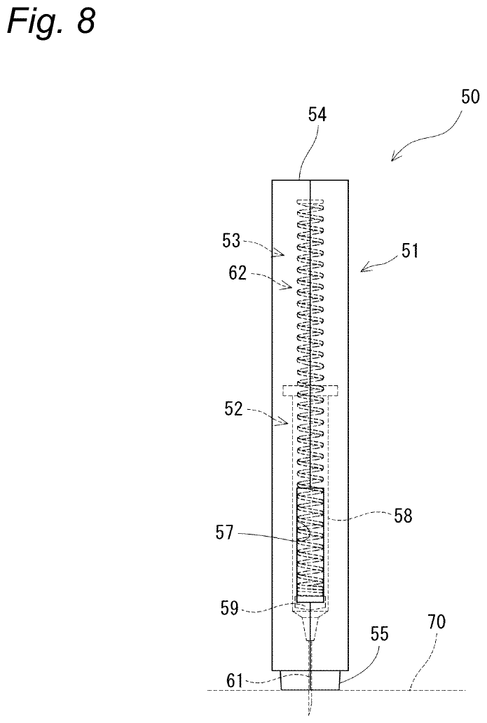

As illustrated in FIGS. 1 to 12, an auto-injector retainer 10 of the present disclosure is capable of retaining a plurality of (herein, two) auto-injectors 50 (see FIGS. 7 and 8) each having a cylindrical housing 51 that houses a prefilled syringe 52 and a gasket pushing mechanism 53, and the auto-injector retainer 10 is used for assisting simultaneous administration by the plurality of auto-injectors 50.

The auto-injector retainer 10 includes a plurality of (herein, two) retaining units 11, 11 capable of retaining the auto-injectors 50 and placed in parallel. The retaining units 11, 11 each include a casing 12 that covers a proximal end of the housing 51 of each auto-injector 50 from outside to house the proximal end. The casing 12 includes a distal opening 13 into which the proximal end of the housing 51 is inserted, a proximal wall 14 abutting on or facing a proximal end face 54 of the housing 51, and a cylindrical peripheral wall 15 elastically deformed when the proximal end of the housing 51 is housed in the casing 12 to hold the proximal end of the housing 51 housed in the casing 12 by an elastic restoring force.

A drug administration device of the present disclosure includes the auto-injector retainer 10 and at least two auto-injectors 50.

With reference to embodiments illustrated in the drawings, hereinafter first described is the outline of an auto-injector used in this embodiment. Various known auto-injectors are employable, and details of each auto-injector 50 will be omitted herein except for a predetermined configuration. In the auto-injector 50 of this embodiment, the prefilled syringe 52 provided with an injection needle 61 and the gasket pushing mechanism 53 are housed in the cylindrical housing 51.

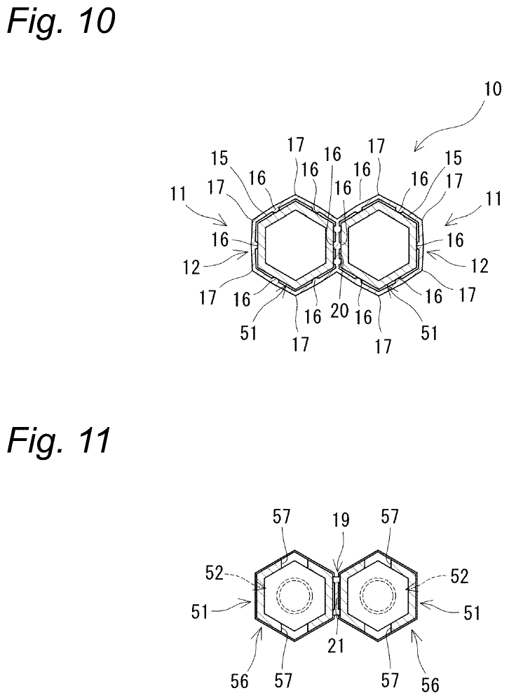

The housing 51 of the auto-injector 50 is a cylindrical member formed of hard or semi-hard resin, having a substantially hexagonal cross section perpendicular to the axial direction as illustrated in FIGS. 10 and 11. The shape of a cross section perpendicular to the axial direction of a housing of an auto-injector may be, for example, circle, rounded polygon, or rounded square with each side curved outward as well as the illustrated shape.

A proximal end (upper end in FIG. 7) of the housing 51 of the auto-injector 50 is closed, and an end face of the proximal end (face on the proximal side) is the proximal end face 54 of the housing 51. A distal end (lower end in FIG. 7) of the housing 51 is opened and provided with a pressing portion 55 (needle cover) protruding from the housing 51 as illustrated in FIGS. 7 and 8. The pressing portion 55 is movable relative to the housing 51 within a predetermined range. As illustrated in FIG. 7, a cap 56 is detachably attached to the auto-injector 50 in order to prevent the pressing portion 55 and the injection needle 61 from being touched from outside before use.

The auto-injector 50 is provided with a window 57 that is formed in a side wall of the housing 51 and used for visually checking the inside of the prefilled syringe 52. A typical auto-injector includes a window for visually checking the inside of a prefilled syringe, specifically, the movement of a gasket housed inside the prefilled syringe. Checking the movement of the gasket from this window enables an understanding of progress and completion of medical liquid administration.

As illustrated in FIGS. 7 and 8, the window 57 of the auto-injector 50 is typically a substantially rectangular through hole formed in the side wall of the housing 51. Accordingly, the inside of the housing 51 can be visually checked from outside of the auto-injector 50 through the window 57. The window 57 is formed in a distal portion of the auto-injector 50. Accordingly, the inside of the prefilled syringe 52 housed in the housing 51 (more specifically, the movement of a gasket 59 in the prefilled syringe 52) can be visually checked from outside of the auto-injector 50 through the window 57. Two windows 57 are formed in the side wall of the housing 51 at positions facing each other (see FIG. 11. Note that FIG. 7 shows only one window). The shape, position, and number of windows are not limited to those illustrated above. For example, the number of windows may be one or three or more. However, from a viewpoint of visibility of the inside of a prefilled syringe through a window, it is preferable that windows are formed in a side wall of a housing at least at positions facing each other.

The prefilled syringe 52 is housed inside the housing 51. The prefilled syringe 52 includes a barrel 58, the gasket 59 slidably housed in the barrel 58 in a liquid-tight manner, and a medical liquid 60 filled in the barrel 58.

The barrel 58 is a transparent or translucent cylindrical member. A proximal end (upper end in FIG. 7) of the barrel 58 (barrel body) is provided with an outward protruding flange. A distal end (lower end in FIG. 7) of the barrel 58 is provided with a nozzle having a diameter decreasing in the distal direction, and the metallic hollow injection needle 61 is attached to the nozzle. The inside and outside of the barrel 58 are communicated with each other through the injection needle 61. A distal end of the injection needle 61 is a puncture needle tip. The barrel 58 is housed (fixed) inside the housing 51 so as not to move relative to the housing 51. Examples of the material for the barrel 58 include glass and plastic.

The gasket 59 is housed in the barrel 58 (barrel body). The gasket 59 is movable, being in contact with the inner surface of the barrel 58 in a liquid-tight manner. A preferable example of the material for the gasket 59 includes rubber having elasticity.

The inside of the prefilled syringe 52, specifically, a space defined by the inner surface of the barrel 58 and the gasket 59 inside the barrel 58 is filled with the medical liquid 60. The medical liquid 60 filled in the prefilled syringe 52 is, for example, a solution, gel, or suspension containing a drug. An employable drug is not substantially limited as long as it is not a drug unsuitable for transdermal administration.

Examples of the drug include a protein preparation, an antibody preparation, hyaluronic acid, an antibacterial drug, an antiviral drug, a vaccine, an antitumor drug, an immunosuppressant, a steroid, an anti-inflammatory drug, an antirheumatic drug, an arthritis therapeutic drug, an antihistamine, an antiallergic drug, a diabetes therapeutic drug, a hormone agent for growth hormone, a bone calcium metabolic drug, a vitamin, a blood preparation, a hematopoietic drug, an antithrombotic drug, an antihyperlipidemic drug, an antiarrhythmic drug, a vasodilator, a prostaglandin, a calcium antagonist, an ACE inhibitor, a β-blocker, an antihypertensive, a diuretic, a xanthine derivative, a β-agonist, an anti-asthmatic drug, a cough suppressant, an expectorant, an anticholinergic drug, an anti-diarrheal drug, a gastrointestinal drug, an antiulcer drug, a purgative, a sleep medication, a sedative, an antipyretic, a cold medication, an antiepileptic drug, an antipsychotic, an antidepressant, an antianxiety drug, a central nervous system stimulant, a parasympathomimetic, a sympathomimetic, an antiemetic, a central stimulant, an anti-parkinsonian drug, a muscle relaxer, an antispasmodic, an anesthetic, an antipruritic, an anti-migraine drug, an oligonucleotide, and a genetic disease drug.

The gasket pushing mechanism 53 is housed inside the housing 51. The detailed structure of the gasket pushing mechanism 53 is omitted herein, but note that, for example, the gasket pushing mechanism 53 includes a coil spring 62 capable of pushing the gasket 59 as illustrated in FIGS. 7 and 8. The coil spring 62 is compressed in advance and restrained from exerting its biasing force before use of the auto-injector 50. The gasket pushing mechanism 53 is actuated by a predetermined operation with respect to the auto-injector 50 (by changing the state of the auto-injector 50 to a predetermined state). In other words, the predetermined operation actuates the gasket pushing mechanism 53 to push (bias) the gasket 59 by the coil spring 62, thereby moving the gasket 59 inside the barrel 58 toward the distal direction of the prefilled syringe 52 (barrel 58).

The actuation of the auto-injector 50 of this embodiment will now be described briefly. When using the auto-injector 50, first, the cap 56 is detached from the auto-injector 50. Next, the housing 51 is gripped and the pressing portion 55 is pressed against an administration site 70, and the housing 51 is advanced toward the administration site 70. Accordingly, as illustrated in FIG. 8, the pressing portion 55 moves inside the housing 51, and the injection needle 61 is exposed (protrudes) from the pressing portion 55, whereby the administration site 70 is punctured with the injection needle 61. This operation (allowing a predetermined length of the pressing portion 55 to enter the housing 51) actuates the gasket pushing mechanism 53. In a state where the administration site 70 is punctured with the injection needle 61, the movement of the gasket 59 in the distal direction of the prefilled syringe 52 (barrel 58) by the gasket pushing mechanism 53 (coil spring 62) causes ejection of the medical liquid 60 from the injection needle 61, whereby the medical liquid 60 is administered to a patient. After the administration of the medical liquid 60, the auto-injector 50 pressed against the administration site 70 is released and the housing 51 is retracted, whereby the pressing portion 55 returns to the state before use and the injection needle 61 is housed again in the housing 51 (pressing portion 55).

The auto-injector 50 of this embodiment is what is called a two-step auto-injector in which the gasket pushing mechanism 53 is automatically actuated by advancing the housing 51 (by allowing a predetermined length of the pressing portion 55 to enter the housing 51) in a state where the pressing portion 55 is pressed against the administration site 70, thereby administering the medical liquid 60 to a patient. What is called a three-step auto-injector may also be employed in which a gasket pushing mechanism is actuated by a predetermined operation (for example, by pushing a separate actuation button) in a state where the administration site 70 of a patient is punctured with an injection needle.

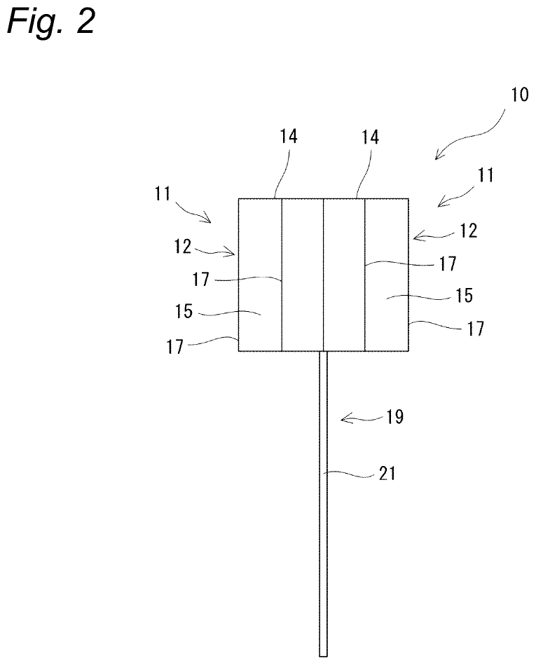

As illustrated in FIGS. 1 to 6, the auto-injector retainer 10 of this embodiment includes the plurality of retaining units 11 capable of retaining the auto-injectors 50 and placed in parallel. In this embodiment, the auto-injector retainer 10 includes two retaining units 11, 11. As illustrated in FIGS. 2 to 6, the two retaining units 11, 11 are bilaterally symmetrical and have similar configurations. Hereinafter, one retaining unit 11 will be described unless otherwise specified.

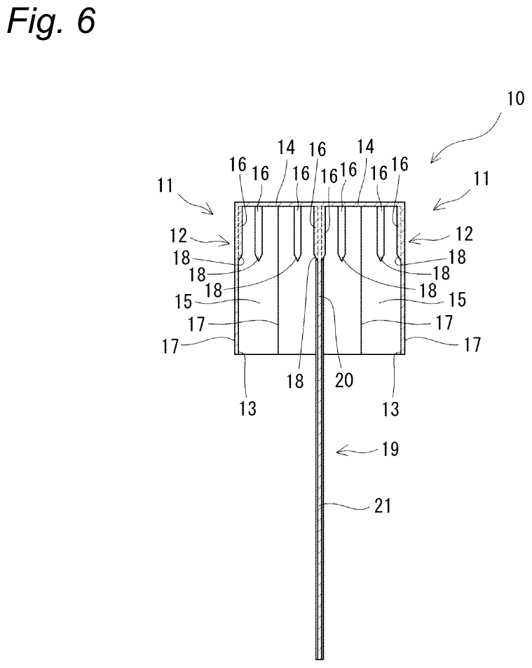

The retaining unit 11 includes the casing 12 that covers the proximal end of the housing 51 of the auto-injector 50 from outside to house the proximal end. As illustrated in FIGS. 2 to 6, the casing 12 includes the distal opening 13, the proximal wall 14, and the cylindrical peripheral wall 15. In other words, in the casing 12, an end (upper end in FIG. 6) on one side (the proximal side) in the axial direction of the cylindrical peripheral wall 15 is closed by the proximal wall 14, and an end (lower end in FIG. 6) on the other side (the distal side) in the axial direction of the cylindrical peripheral wall 15 is opened as the distal opening 13.

The internal shape (inner edge shape, interior configuration) of the distal opening 13 corresponds to the external shape (exterior configuration) of the proximal end of the housing 51 of the auto-injector 50 (herein, a substantially hexagonal shape). In addition, the internal shape of the distal opening 13 is similar to or slightly larger than the external shape of the proximal end of the housing 51 of the auto-injector 50, and the proximal end of the housing 51 is inserted from the distal opening 13. The distal opening 13 allows the proximal end of the housing 51 to enter the casing 12 (the cylindrical peripheral wall 15).

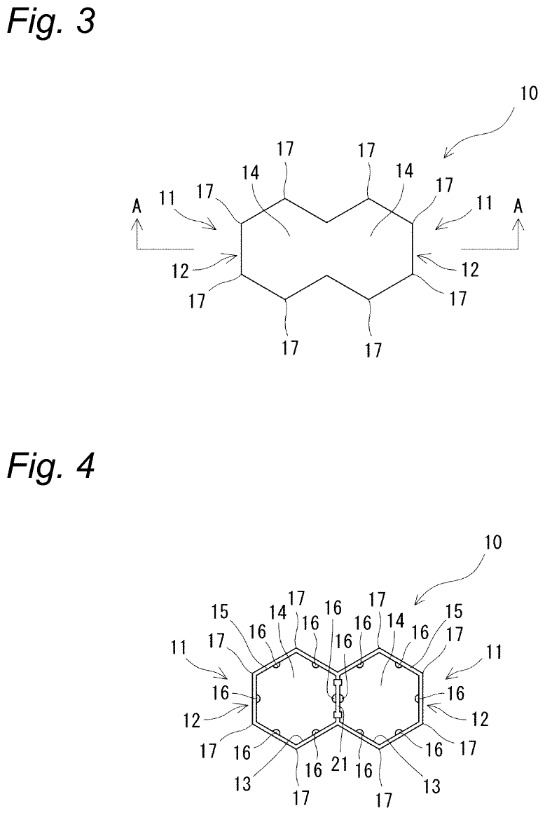

The proximal wall 14 is disposed at a proximal end of the cylindrical peripheral wall 15. In this embodiment, proximal walls 14, 14 of two casings 12, 12 are plates that close the proximal end of each cylindrical peripheral wall 15, and the two proximal walls 12, 12 form one continuous wall (plate) as illustrated in FIG. 3. The proximal wall 14 abuts on or faces the proximal end face 54 of the housing 51 of the auto-injector 50 that is allowed to enter the casing 12 (cylindrical peripheral wall 15) from the distal opening 13. This configuration restricts the movement of the auto-injector 50 in the proximal direction. Furthermore, bringing proximal end faces 54, 54 of housings 51, 51 of the auto-injectors 50, 50 into contact with the proximal walls 14, 14 enables alignment of proximal ends of the two auto-injectors 50, 50.

The cylindrical peripheral wall 15 has a predetermined internal (cross sectional) shape (herein, as illustrated in FIG. 4, a substantially hexagonal shape) and has a predetermined length extending in the axial direction (vertical direction in FIG. 2). In this embodiment, the cylindrical peripheral wall 15 has a polygonal cylindrical shape, specifically, a hexagonal cylindrical shape. The cylindrical peripheral wall 15 has a substantially constant plate thickness except for portions provided with ribs 16 to be described. Note that the internal (cross sectional) shape of a cylindrical peripheral wall of an auto-injector retainer is not limited to the illustrated shape (a substantially hexagonal shape). In addition to a polygonal shape, the internal shape may be, for example, circle, rounded polygon, or rounded square with each side curved outward. Furthermore, the internal (cross sectional) shape of a cylindrical peripheral wall of an auto-injector retainer preferably corresponds to the shape of a housing (external shape) of an auto-injector to be retained.

The cylindrical peripheral wall 15 is elastically deformed when the proximal end of the housing 51 is housed in the casing 12 and holds the proximal end of the housing 51 by an elastic restoring force. In this embodiment, although the internal shape of the cylindrical peripheral wall 15 is slightly larger than the external shape of the proximal end of the housing 51 of the auto-injector 50, the proximal end of the housing 51 housed in the casing 12 can be held by providing the cylindrical peripheral wall 15 with the ribs 16 to be described.

Specifically, as illustrated in FIGS. 4 and 6, the cylindrical peripheral wall 15 has the inner surface provided with the ribs 16 protruding inward and abutting on the outer surface of the proximal end of the housing 51 housed in the casing 12. In this embodiment, the ribs 16 included in the cylindrical peripheral wall 15 extend in the axial direction of the cylindrical peripheral wall 15.

More specifically, as illustrated in FIGS. 4 and 6, the ribs 16 formed on the inner surface of the cylindrical peripheral wall 15 have a substantially semicircular cross section and extend in the proximal direction from an axially central portion of the cylindrical peripheral wall 15 to the proximal end of the cylindrical peripheral wall 15 (up to the proximal wall 14). In this embodiment, as illustrated in the cross sections illustrated in FIGS. 4 and 10, one rib 16 is disposed on each side of a hexagon forming the inner surface of the cylindrical peripheral wall 15 (each face of the cylindrical peripheral wall 15), and a total of six ribs 16 are disposed on the cylindrical peripheral wall 15. Each rib 16 is preferably disposed on a central portion of each face in the cylindrical peripheral wall 15 (a portion of the cylindrical peripheral wall 15 excluding a corner 17, that is, a central portion between adjacent corners 17, 17). The central portion (the central portion of each face in the cylindrical peripheral wall 15) is elastically deformed more easily than the corner 17, and portions of the cylindrical peripheral wall 15 provided with the ribs 16 are easy to deform elastically. In addition, the ribs 16 do not excessively increase force required for elastically deforming the cylindrical peripheral wall 15 and do not inhibit the movement of the auto-injector 50 in the proximal direction inside the cylindrical peripheral wall 15 (casing 12). Note that ribs are not necessarily disposed on all faces of a cylindrical peripheral wall (all faces of a polygonal cylindrical inner surface) and may be disposed on selected faces.

A distal end of each rib 16 is provided with an inclined portion 18 protruding and tapering toward the distal direction. The inclined portion 18 guides the proximal-ward movement of the proximal end of the housing 51 inserted into (allowed to enter) the casing 12 from the distal opening 13. In addition, the outer surface of the proximal end of the housing 51 abuts on the ribs 16 from each inclined portion 18, whereby the cylindrical peripheral wall 15 is gradually elastically deformed.

As illustrated in FIG. 10, in a state where the auto-injector 50 is housed, each rib 16 abuts on the outer surface of the proximal end of the housing 51. When each rib 16 abuts on the outer surface of the proximal end of the housing 51, the cylindrical peripheral wall 15 is elastically deformed, and the proximal end of the housing 51 is held by an elastic restoring force. Accordingly, it is possible to maintain an axial position of the auto-injector 50 and to prevent the auto-injector 50 from being unintentionally detached from the casing 12.

In this embodiment, when each rib 16 abuts on the outer surface of the proximal end of the housing 51, a gap (space) is formed between the inner surface of the cylindrical peripheral wall 15 excluding the portions provided with the ribs 16 and the outer surface of the proximal end of the housing 51. In addition, in this embodiment, each rib 16 abuts on the outer surface of the proximal end of the housing 51, that is to say, the inner surface of the cylindrical peripheral wall 15 does not entirely abut on the outer surface of the proximal end of the housing 51. Therefore, a contact area between the outer surface of the proximal end of the housing 51 and the inner surface of the cylindrical peripheral wall 15 is small and frictional resistance is also small as compared with a case where the outer surface of the proximal end of the housing 51 and the entire inner surface of the cylindrical peripheral wall 15 are brought into contact with each other. This configuration provides the advantage that the movement of the auto-injector 50 in the axial direction (proximal direction) is facilitated inside the casing 12 (cylindrical peripheral wall 15).

While the casing 12 (cylindrical peripheral wall 15) houses the proximal end of the housing 51 of the auto-injector 50, it is preferable that the casing 12 does not cover the window 57 formed in the housing 51. In other words, it is preferable that the casing 12 (cylindrical peripheral wall 15) is not extended to a part where the window 57 is formed. Accordingly, the casing 12 (cylindrical peripheral wall 15) does not impede the visibility of the inside of the prefilled syringe through the window 57.

The auto-injector retainer 10 includes a partition unit 19 for maintaining the auto-injectors 50, 50 retained by two adjacent retaining units 11, 11 in a state of mutual separation. The partition unit 19 is a part of the casings 12, 12 in the two adjacent retaining units 11, 11 and includes a casing partition 20 having a predetermined thickness. In addition, the partition unit 19 includes an extending partition 21 extending from distal ends (lower ends in FIG. 2) of the retaining units 11, 11, having a predetermined thickness, and placed between the auto-injectors 50, 50 retained by the two adjacent retaining units 11, 11. In this embodiment, the casing partition 20 and the extending partition 21 continuously extend in an integrated manner and have a cross-section with substantially the same shape (herein, a substantially I-shaped cross section in a plane perpendicular to the axial direction of the cylindrical peripheral wall 15).

Specifically, as illustrated in FIGS. 4 and 10, in the two adjacent retaining units 11, 11 (the casings 12, 12), a wall of one casing 12 closer to the other casing 12 (a part of the cylindrical peripheral wall 15) is combined to a wall of the other casing 12 closer to one casing 12 (to form a common partition), and a part of the walls and both ends thereof protruding toward the inside of each cylindrical peripheral wall 15 (upper and lower ends in FIG. 10) form the casing partition (common partition) 20 having a substantially I-shaped cross section in a plane perpendicular to the axial direction of the cylindrical peripheral wall 15.

The casing partition 20 is formed between the two casings 12, 12 and placed between the two auto-injectors 50, 50 in a state where the two auto-injectors 50, 50 are retained by the auto-injector retainer 10. With the casing partition 20, as illustrated in FIG. 10, the proximal ends of the auto-injectors 50, 50 retained by the two adjacent retaining units 11, 11 are maintained at a predetermined interval, and injection needles 61, 61 of the auto-injectors 50, 50 are maintained at a predetermined interval. Accordingly, administration sites (injection sites) of a medical liquid to be administered by a plurality of auto-injectors are prevented from getting too close, and it is also possible to prevent problems caused by such closely situated administration sites.

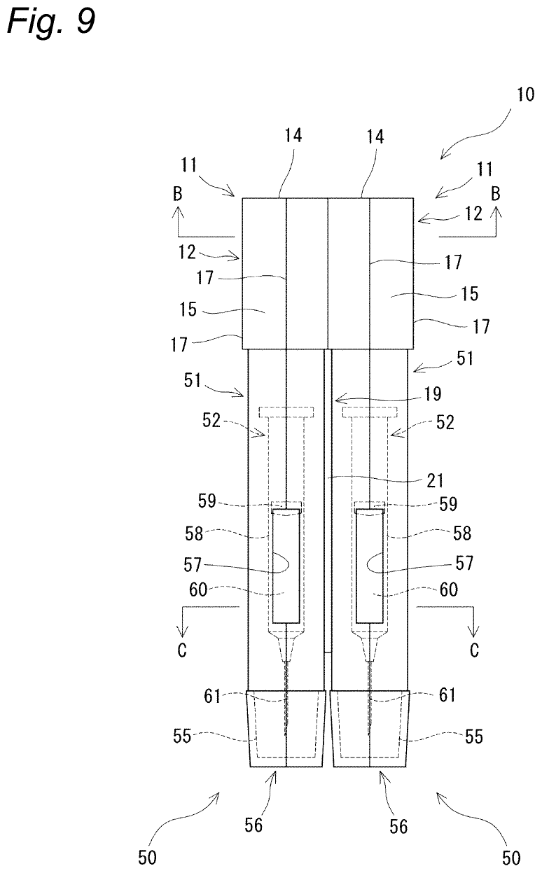

Furthermore, in this embodiment, as illustrated in FIGS. 4 and 11, the extending partition 21 is substantially equal to the casing partition 20 in cross-sectional shape (has a substantially I-shaped cross section in a plane perpendicular to the axial direction of the cylindrical peripheral wall 15) and extends in the distal direction from the distal ends (lower ends in FIG. 2) of the retaining units 11. As illustrated in FIGS. 9 and 11, in a state where the two auto-injectors 50, 50 are retained by the auto-injector retainer 10, the extending partition 21 is placed between the two auto-injectors 50, 50. The extending partition 21 maintains a predetermined interval between the two adjacent auto-injectors 50, 50, particularly, between the injection needles 61, 61 of the auto-injectors 50, 50. Accordingly, administration sites (injection sites) of a medical liquid to be administered by a plurality of auto-injectors are prevented from getting too close, and it is also possible to prevent problems caused by such closely situated administration sites.

Particularly, in this embodiment, the retaining unit 11 (casing 12) of the auto-injector retainer 10 retains (houses) the proximal end of the auto-injector 50. For this reason, when a patient grips distal portions of the two auto-injectors 50, 50 (a portion of each auto-injector 50 closer to the distal end than a portion housed in the casing 12) at the time of administration, the interval between the distal portions narrows, and the distal ends (injection needles 61, 61) of the auto-injectors 50, 50 may move in directions in which they approach each other. The extending partition 21 maintains the distal portions of the two adjacent auto-injectors 50, 50 at a predetermined interval, thereby maintaining the injection needles 61, 61 of the auto-injectors 50, 50 at a predetermined interval more reliably.

A preferable material for the auto-injector retainer 10 is hard or semi-hard resin that is elastically deformable to some extent. Examples of the material include polyvinyl fluoride, polycarbonates, acrylic resins (such as polyacrylates (specifically, polymethyl methacrylate, or PMMA), polyacrylamides, acrylonitrile-styrene copolymers, and acrylonitrile-butadiene-styrene copolymers), polyesters (such as polyethylene terephthalate and polybutylene terephthalate), polyolefins (such as polyethylene, polypropylene, and ethylene-propylene copolymers), styrene-based resins (such as polystyrene, MS resin (methacrylate-styrene copolymers), and MBS resin (methacrylate-butylene-styrene copolymers)), polyvinyl chloride (hard vinyl chloride), and polyamides (such as Nylon 6 and Nylon 66). Alternatively, examples of the material for the auto-injector retainer 10 include rubbers having elasticity (for example, butyl rubber, latex rubber, silicone rubber, styrene-butadiene rubber, and isoprene) and thermoplastic elastomers (for example, styrene-based elastomers such as SBS elastomer, SEBS elastomer, and SEPS elastomer and olefin-based elastomers such as ethylene-α-olefin copolymer elastomer).

Hereinafter briefly described is simultaneous administration of a medical liquid by a plurality of (herein, two) auto-injectors 50 using the auto-injector retainer 50 of this embodiment.

First, as illustrated in FIGS. 9 to 11, the retaining units 11, 11 of the auto-injector retainer 10 respectively retain the auto-injectors 50. It is preferable that the window 57 of each auto-injector 50 is set at a position visible from outside (windows 57, 57 of the two auto-injectors 50, 50 are not facing each other).

In addition, each auto-injector 50 (the proximal end of the housing 51) are preferably pushed into the casing 12 (the cylindrical peripheral wall 15) with some degree of force (specifically, a force larger than a force required for puncturing the after-mentioned administration site 70 with the injection needle 61). Accordingly, it is possible to prevent relative displacement of the auto-injector retainer 10 and the auto-injectors 50 during the administration.

It is preferable that the two auto-injectors 50, 50 are at substantially the same axial position. In this embodiment, the two auto-injectors 50, 50 are of the same type, and the two retaining units 11, 11 have substantially the same configuration. Accordingly, when each auto-injector 50 (the proximal end of the housing 51) is allowed to enter the casing 12 (cylindrical peripheral wall 15) by substantially the same force, the two auto-injectors 50, 50 are placed at substantially the same axial position. Furthermore, each auto-injector 50 may enter the casing 12 (the cylindrical peripheral wall 15) until the proximal end face 54 abuts on the proximal wall 14. Accordingly, it is possible to align the proximal ends of the two auto-injectors 50, 50.

While the two auto-injectors 50, 50 are retained by the auto-injector retainer 50, aligning the two auto-injectors 50, 50 at substantially the same axial position makes it possible to retain a distal end (cutting edge) of each injection needle 61 and a distal end of the pressing portion 55 at substantially the same position with respect to the axial direction of the auto-injector retainer 50.

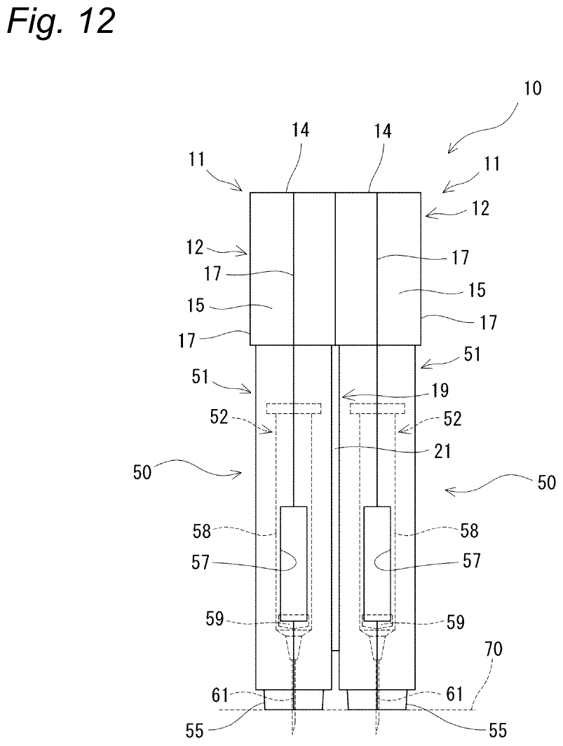

Next, as illustrated in FIG. 12, the cap 56 is detached from each auto-injector 50, and the auto-injectors 50, 50 and/or the auto-injector retainer 10 are gripped and the pressing portions 55, 55 are pressed against the administration site 70. At this time, the proximal end of the housing 51 of each of the auto-injectors 50, 50 is held by the retaining unit 11 (casing 12) (or retained substantially immovable in the axial direction relative to the retaining unit 11), and the two auto-injectors 50, 50 advance toward the administration site 70 substantially simultaneously. In addition, the partition unit 19 (the casing partition 20 and the extending partition 21) is placed between the two auto-injectors 50, 50. Therefore, even when any part of the auto-injectors 50, 50 and/or the auto-injector retainer 10 are gripped, the auto-injectors 50, 50 are maintained at a predetermined interval, and the injection needles 61, 61 of the auto-injectors 50, 50 are maintained at a predetermined interval.

Each pressing portion 55 moves inside the housing 51, and the injection needles 61 are exposed (protrude) from the pressing portions 55, whereby the administration site 70 is punctured with the injection needles 61. This operation actuates the gasket pushing mechanism 13. The gasket pushing mechanism 13 moves the gasket 59 in the distal direction of the prefilled syringe 52 (barrel 58), and the medical liquid 60 is ejected from the injection needle 61, thereby administering the medical liquid 60 to a patient (see FIG. 12). As illustrated in FIGS. 9 and 12, a position (distal-ward movement) of the gasket 59 inside the prefilled syringe 52 can be visually checked through the window 57. Accordingly, it is possible to determine from outside that the medical liquid 60 has been appropriately administered by the auto-injectors 50 retained by the auto-injector retainer 10.

As described above, the auto-injector retainer of the present invention (auto-injector retainer 10) is capable of appropriately bundling and retaining a plurality of auto-injectors 50 and appropriately assisting simultaneous administration of a medical liquid by the plurality of auto-injectors 50 without inhibiting functions of the auto-injectors 50.

In addition, the auto-injector retainer of the present invention (auto-injector retainer 10) holds the proximal ends of the auto-injectors 50 by an elastic restoring force of the cylindrical peripheral wall 15, thereby hardly causing breakage and deformation (plastic deformation) of the auto-injectors 50 which are attributed to the retaining of the auto-injectors 50. Therefore, the auto-injector retainer of the present invention is suitable for repeated use or use outside one's house (where there is no alternative auto-injector retainer).

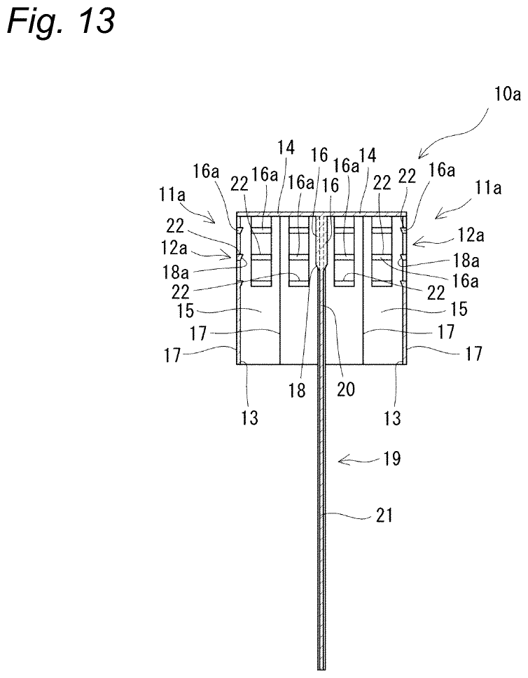

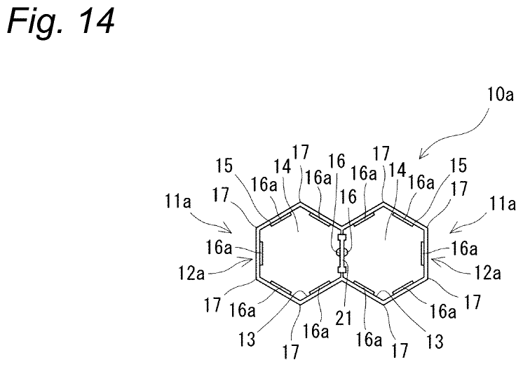

Ribs included in a cylindrical peripheral wall may be ribs extending in the circumferential direction of a cylindrical peripheral wall or ribs distributed on the inner surface of the cylindrical peripheral wall. For example, in an auto-injector retainer 10a (retaining unit 11a, casing 12a) illustrated in FIGS. 13 and 14, three ribs 16a extending in the circumferential direction of a cylindrical peripheral wall 15 are formed on each face of the cylindrical peripheral wall 15. A distal portion of each rib 16a is provided with an inclined portion 18a protruding and tapering toward the distal direction.

In the auto-injector retainer 10a, a plurality of openings 22 are formed in the cylindrical peripheral wall 15. Each opening 22 is formed in such a manner that its peripheral edge on the distal side coincides with the proximal edge of each rib 16a. Such an opening 22 is intended to eliminate undercuts of the cylindrical peripheral wall 15 provided with the ribs 16a and to ensure moldability (particularly, during injection molding) of the auto-injector retainer 10a, but forming the opening 22 easily enables elastic deformation of the cylindrical peripheral wall 15.

Other embodiments of the auto-injector retainer of the present invention will be described with reference to the drawings. Note that the following auto-injector retainer is similar to the auto-injector retainer 10 in basic configuration but different in configuration of a retaining unit (casing). Unless otherwise specified, configurations substantially similar to those in the auto-injector retainer 10 are denoted by the same or corresponding names and reference numerals, and details thereof will be omitted.

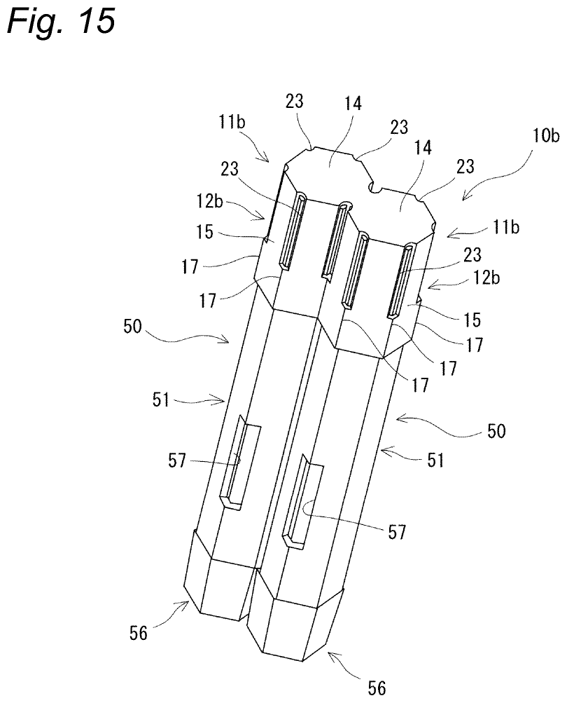

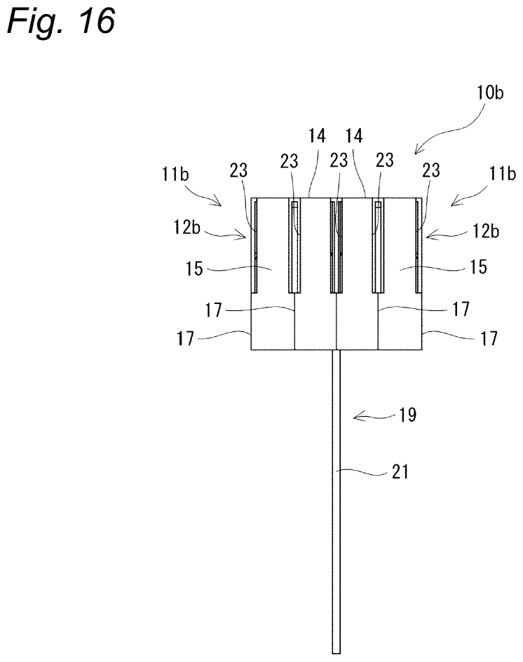

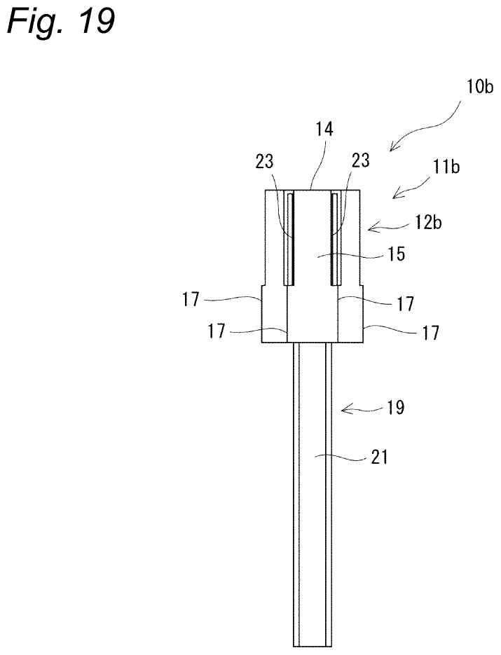

As illustrated in FIGS. 15 to 20, an auto-injector retainer 10b has a cylindrical peripheral wall 15 provided with a plurality of peripheral wall slits 23 extending in the axial direction. The peripheral wall slits 23 extend from a proximal end of the cylindrical peripheral wall 15 toward a distal opening 13 and do not reach the distal opening 13. In this embodiment, the cylindrical peripheral wall 15 is a polygonal cylindrical peripheral wall having a polygonal (herein, a substantially hexagonal) cross section perpendicular to the axial direction, and the peripheral wall slits 23 are formed in corners 17 extending in the axial direction of the polygonal cylindrical peripheral wall (cylindrical peripheral wall 15).

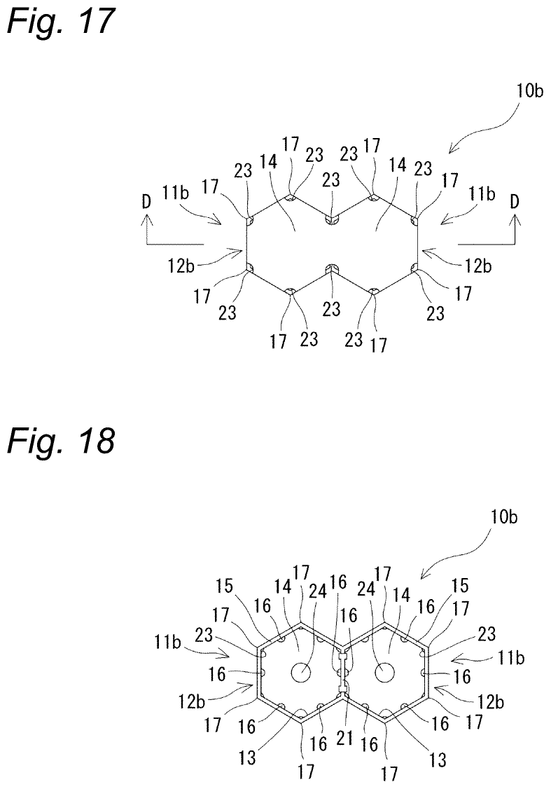

Specifically, as illustrated in FIGS. 17 and 18, the cylindrical peripheral wall 15 of a retaining unit 11b (casing 12b) is a polygonal cylindrical peripheral wall having a substantially hexagonal cross section, and each peripheral wall slit 23 is formed in each of six corners 17 extending in the axial direction. The peripheral wall slits 23 penetrate the cylindrical peripheral wall 15 and communicate the inside and outside of the casing 12b. In this embodiment, at a portion where two adjacent casings 12b, 12b (cylindrical peripheral walls 15, 15) are connected, two peripheral wall slits 23, 23 formed in respective corners 17 are combined. Peripheral wall slits are not necessarily formed in all corners of a cylindrical peripheral wall, or a polygonal cylindrical peripheral wall, and may be formed only in selected corners. However, two corners 17, 17 sandwiching a portion of the cylindrical peripheral wall 15 where a rib 16 is formed (a portion between adjacent corners 17, 17) is preferably provided with the peripheral wall slits 23.

In the auto-injector retainer 10b, the cylindrical peripheral wall 15 is elastically deformed with ease due to the plurality of peripheral wall slits 23, and the proximal end of the housing 51 housed in the casing 12b is appropriately held by an elastic restoring force. Particularly, in this embodiment, the peripheral wall slits 23 are formed in the corners 17 extending in the axial direction of the polygonal cylindrical peripheral wall (cylindrical peripheral wall 15), whereby a portion of the cylindrical peripheral wall 15 between two adjacent peripheral wall slits 23, 23 is deformed elastically in an appropriate manner.

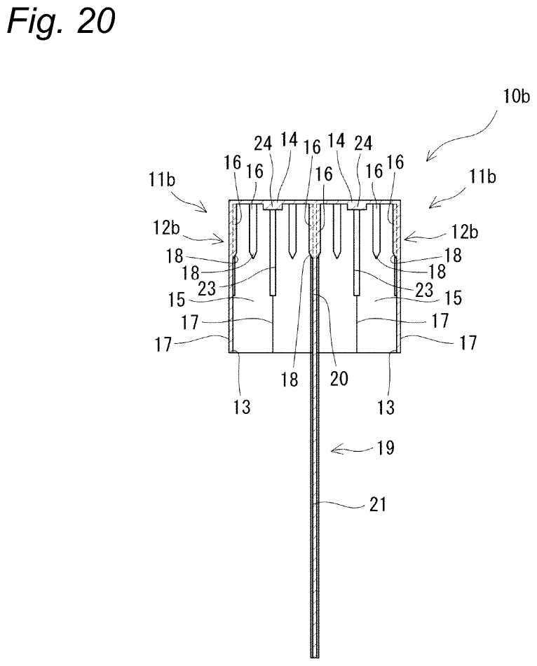

As illustrated in FIG. 20, it is preferable that each peripheral wall slit 23 is equal to or longer than the rib 16 and extends in the distal direction. Accordingly, it is easier to elastically deform the portion of the cylindrical peripheral wall 15 provided with the rib 16.

In addition, as illustrated in FIGS. 18 and 20, a proximal wall 14 of the auto-injector retainer 10b includes an abutting projection 24 protruding inward from a central portion of the inner surface of the casing 12b on the proximal side. Accordingly, even when the proximal end of the housing 51 of the auto-injector 50 is advanced to the proximal end of the casing 12b, the inner surface of the casing 12b on the proximal side has a gap between a portion excluding the abutting projection 24 and the proximal end face 54 of the housing 51 so as not to hinder elastic deformation of the cylindrical peripheral wall 15. In this embodiment, the abutting projection 24 has a columnar shape protruding from the center of the proximal wall 14.

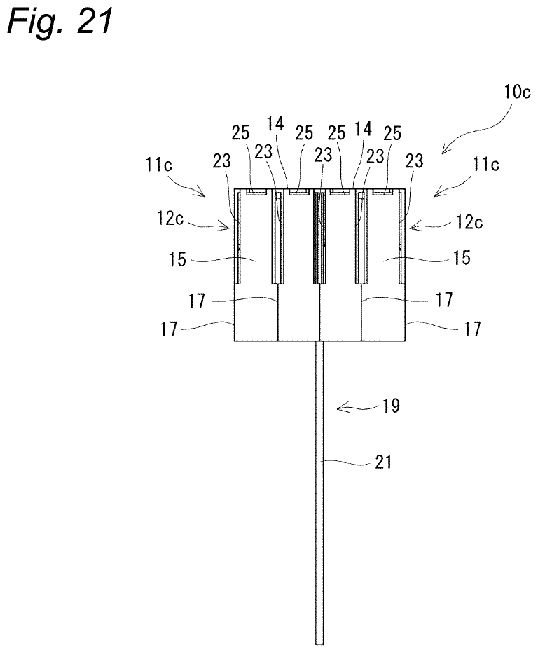

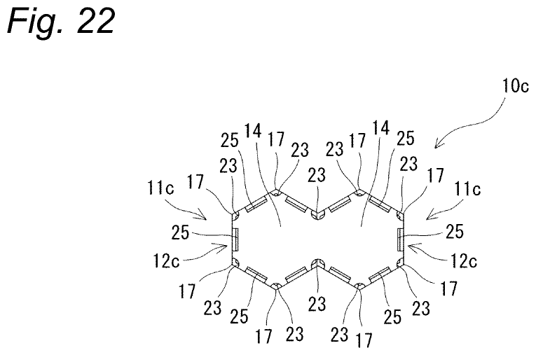

Alternatively, like an auto-injector retainer 10c illustrated in FIGS. 21 and 22, proximal corners may be provided with proximal corner slits 25 (that is, proximal wall side slits). The proximal corners are formed by linear outer edges of a proximal wall 14 and a linear proximal end of a cylindrical peripheral wall 15. The proximal corner slits 25 penetrate the proximal wall 14 and the cylindrical peripheral wall 15, communicating the inside and the outside of a casing 12c. In the auto-injector retainer 10c, the proximal corner slits 25 are formed in a total of ten proximal corners excluding a portion where two adjacent cylindrical peripheral walls 15 are connected. Note that proximal corner slits are not necessarily formed in all proximal corners formed by a cylindrical peripheral wall and a proximal wall and may be formed only in selected proximal corners. However, it is preferable to form a proximal wall slit 25 in a proximal corner corresponding to a portion of the cylindrical peripheral wall 15 provided with a rib 16 (a portion between adjacent corners 17, 17).

Providing the auto-injector retainer 10c with a plurality of proximal corner slits 25 facilitates elastic deformation of the cylindrical peripheral wall 15, which enables the auto-injector retainer 10c to appropriately hold the proximal end of the housing 51 housed in the casing 12c of a retaining unit 11c by an elastic restoring force.

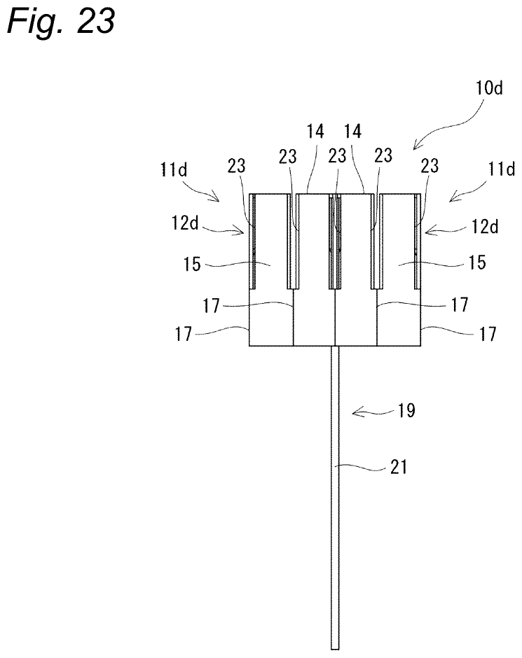

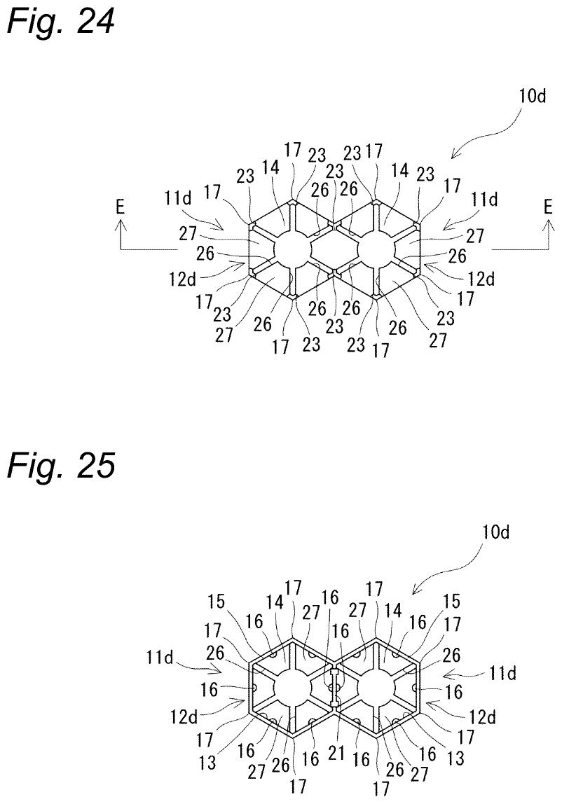

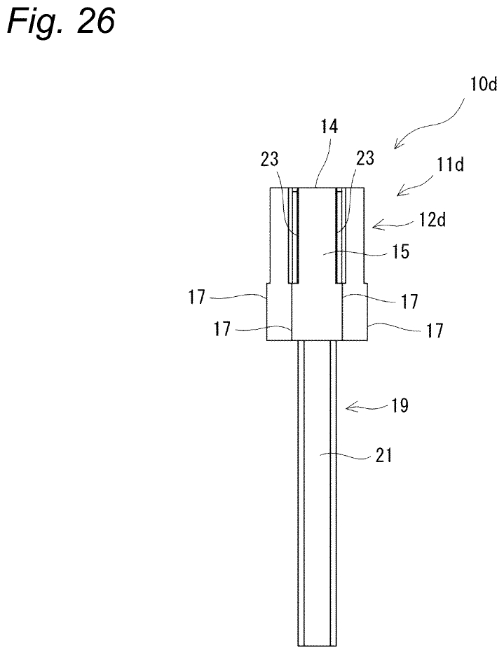

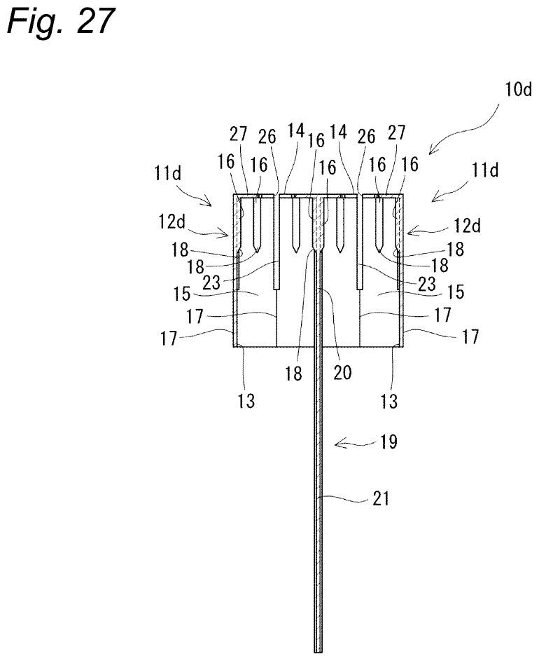

In an auto-injector retainer 10d illustrated in FIGS. 23 to 27, a proximal wall 14 includes a plurality of proximal wall slits 26 continuous with peripheral wall slits 23. The proximal wall 14 is divided into a plurality of proximal segments 27 by the plurality of proximal wall slits 26, and the proximal segments 27 are displaceable along with elastic deformation of the cylindrical peripheral wall 15 continuous with the proximal segments 27. This configuration makes it possible to elastically deform the cylindrical peripheral wall 15 more easily.

In this embodiment, the proximal wall slits 26 extend from all the peripheral wall slits 23 formed in a retaining unit 11d (casing 12d) toward a central portion of the proximal wall 14. The central portion of the proximal wall 14 is cut out, and each proximal wall slit 26 is connected to the central cutout. Accordingly, each proximal wall 14 is divided into six proximal segments 27.

In the auto-injector retainer 10d, the cylindrical peripheral wall 15 (each face of the cylindrical peripheral wall 15) between two adjacent peripheral wall slits 23, 23 and the proximal segments 27 are combined and displaceable (elastically deformable) independently of the other portion of the cylindrical peripheral wall 15 and the proximal segments 27. Accordingly, the overall cylindrical peripheral wall 15 is elastically deformed more easily, and this configuration is employable in a case, for example, where it is desired to retain (hold) the proximal end of the housing 51 with a relatively weak force in the retaining unit 11d. In the auto-injector retainer 10d, the central portion of the proximal wall 14 is cut out and not provided with a configuration corresponding to the abutting projection 24 but ensures the ease of elastic deformation of the cylindrical peripheral wall 15 as described above.

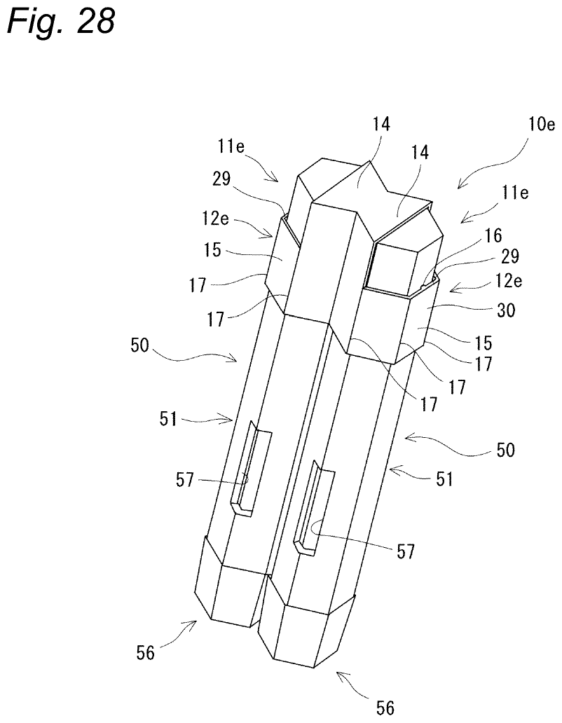

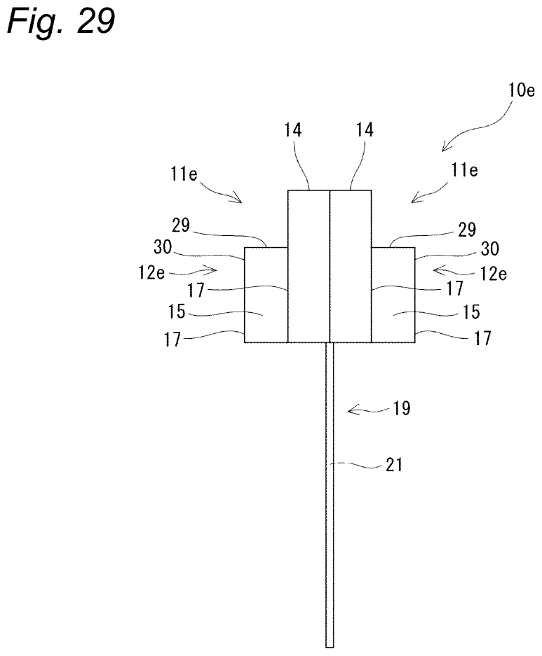

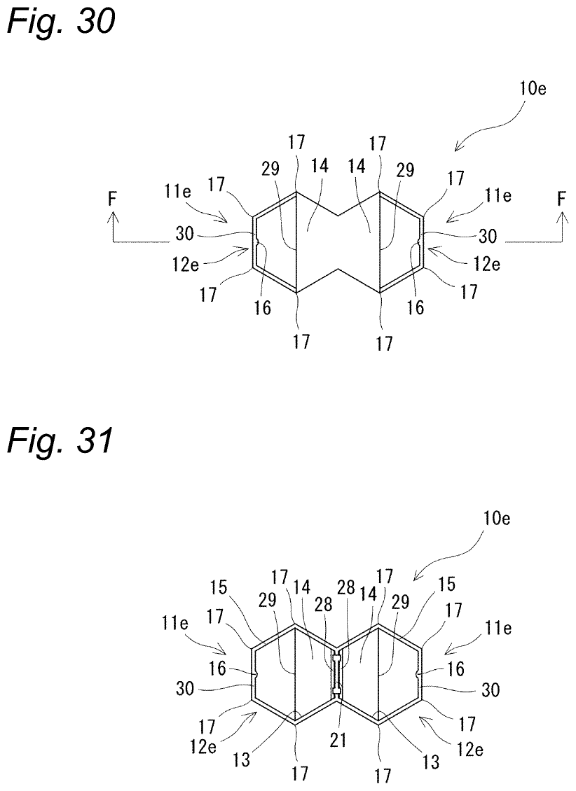

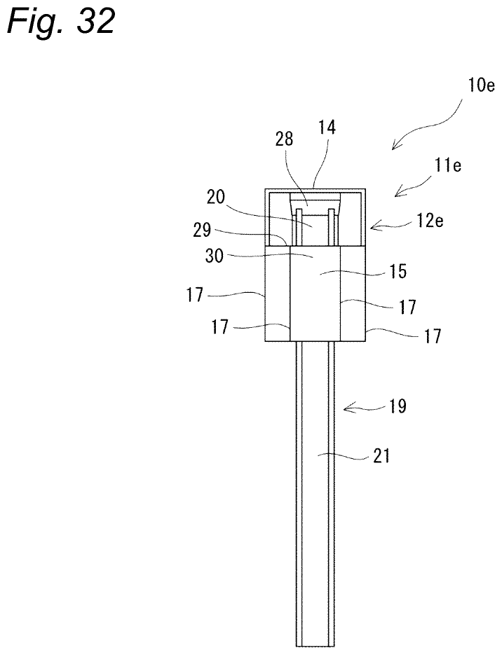

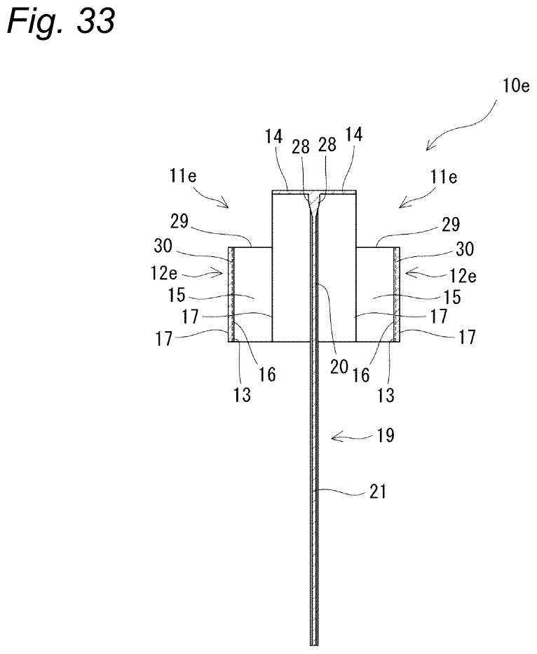

As illustrated in FIGS. 28 to 33, a proximal end of a casing 12e of an auto-injector retainer 10e has an inner part provided with a protrusion 28 protruding inward and abutting on the proximal end of the housing 51 housed in the casing 12e to press the proximal end of the housing 51 housed in the casing 12e toward the inner surface of the opposed cylindrical peripheral wall 15. In other words, the protrusion 28 presses the proximal end of the housing 51 housed in the casing 12e toward the inner surface of the cylindrical peripheral wall 15 that faces the protrusion 28. More specifically, as illustrated in FIG. 33, the protrusion 28 has a distal portion protruding and inclined so as to swell gradually inward from the distal side toward the proximal side. Accordingly, the proximal end of the housing 51 allowed to enter the casing 12e in the proximal direction is gradually pushed in the protruding direction of the protrusion 28.

In addition, the auto-injector retainer 10e includes a proximal cutout 29 formed by cutting out a part of the cylindrical peripheral wall 15 facing the protrusion 28 and a part of the proximal wall 14 continuous to the cylindrical peripheral wall 15, and a portion of the cylindrical peripheral wall 15 facing the protrusion 28 where the proximal cutout 29 is not formed serves as a clamping wall 30 that clamps the proximal end of the housing 51 together with the protrusion 28.

Specifically, as illustrated in FIGS. 28 to 33, a substantially half portion on the proximal side of the cylindrical peripheral wall 15 facing the protrusion 28 is cut out, and a substantially half portion of the proximal wall 14 continuous to the cylindrical peripheral wall 15 is cut out. The proximal cutout 29 is open at a portion of the casing 12e facing the protrusion 28. The portion where the proximal cutout 29 is not formed in the cylindrical peripheral wall 15 facing the protrusion 28 (specifically, obliquely facing the distal side in the axial direction) serves as the clamping wall 30 that clamps the proximal end of the housing 51 together with the protrusion 28. The clamping wall 30 is elastically deformed when the proximal end of the housing 51 is pressed by the protrusion 28 and clamps the proximal end of the housing 51 by its elastic restoring force. In other words, in a retaining unit 11e (the casing 12e), while the proximal cutout 29 allows displacement of the proximal end of the housing 51 pushed by the protrusion 28, the proximal end of the housing 51 is clamped by an elastic restoring force of the clamping wall 30 that is elastically deformed by the displacement.

In the auto-injector retainer 10e, a rib 16 is disposed on a portion (face) of the cylindrical peripheral wall 15 (the clamping wall 30) facing the protrusion 28. In this embodiment, the rib 16 extends from a distal opening 13 to a proximal end (proximal cutout 29) of the clamping wall 30. When the rib 16 abuts on the outer surface of the proximal end of the housing 51, a gap (space) is formed between the inner surface of the cylindrical peripheral wall 15 (the clamping wall 30) excluding the portion provided with the rib 16 and the outer surface of the proximal end of the housing 51. With this configuration, the cylindrical peripheral wall 15 (the clamping wall 30) is not inhibited from deforming elastically. In this embodiment, the rib 16 is not disposed on other portions (faces) of the cylindrical peripheral wall 15.

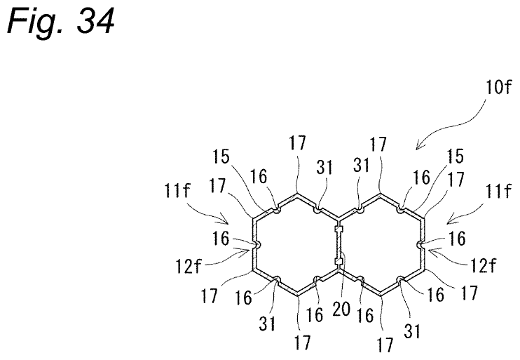

Like an auto-injector retainer 10f illustrated in FIG. 34, a recess 31 may be disposed on the outer surface of a cylindrical peripheral wall 15, corresponding to a portion where a rib 16 is formed. The recess 31 appropriately adjusts the ease of elastic deformation of the cylindrical peripheral wall 15 in a retaining unit 11f (casing 12f) provided with the rib 16.

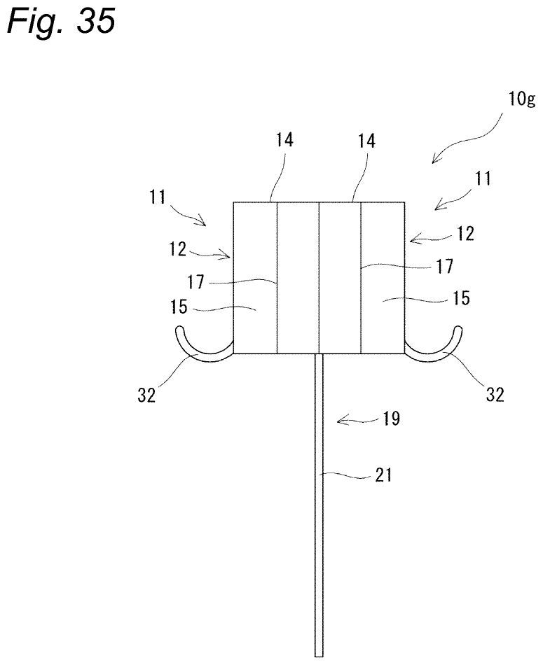

Alternatively, like an auto-injector retainer 10g illustrated in FIG. 35, an auto-injector retainer may be provided with a handle 32 that is gripped during administration. Gripping the handle 32 of the auto-injector retainer 10g retaining a plurality of auto-injectors 50 enables administration while maintaining axial positions of the plurality of auto-injectors 50.

Any of the above embodiments employs the same type of auto-injectors as the plurality of auto-injectors, but the auto-injectors may be of different types. In a case where auto-injectors have proximal ends with different shapes, the configuration of each retaining unit of an auto-injector retainer is designed according to the shape of each auto-injector.

The auto-injector retainer of the present invention includes a plurality of retaining units capable of retaining auto-injectors and placed in parallel. Each retaining unit includes a casing that covers a proximal end of a housing of each auto-injector from outside to cover the proximal end. The casing includes a distal opening into which the proximal end of the housing is inserted, a proximal wall abutting on or facing a proximal end face of the housing, and a cylindrical peripheral wall elastically deformed when the proximal end of the housing is housed in the casing to hold the proximal end of the housing housed in the casing by an elastic restoring force. Accordingly, the auto-injector retainer of the present invention appropriately bundles and retains a plurality of auto-injectors and appropriately assists simultaneous administration of a medical liquid by the plurality of auto-injectors without inhibiting functions of the auto-injectors.

The drug administration device of the present invention includes the auto-injector retainer and at least two auto-injectors. Accordingly, the auto-injector retainer of the present invention appropriately bundles and retains a plurality of auto-injectors and appropriately assists simultaneous administration of a medical liquid by the plurality of auto-injectors without inhibiting functions of the auto-injectors.

### INDUSTRIAL APPLICABILITY

An auto-injector retainer of the present invention is as follows.
(1) An auto-injector retainer capable of retaining a plurality of auto-injectors each having a housing that has a cylindrical shape and houses a prefilled syringe and a gasket pushing mechanism, the auto-injector retainer being used for assisting simultaneous administration by the plurality of auto-injectors, and the auto-injector retainer including:
a plurality of retaining units capable of retaining the auto-injectors and placed in parallel,
wherein the retaining units each include a casing that covers a proximal end of the housing of each of the auto-injectors from outside to house the proximal end, and
the casing includes a distal opening into which the proximal end of the housing is inserted, a proximal wall abutting on or facing a proximal end face of the housing, and a cylindrical peripheral wall elastically deformed when the proximal end of the housing is housed in the casing to hold the proximal end of the housing housed in the casing by an elastic restoring force.

This auto-injector retainer includes the plurality of retaining units capable of retaining auto-injectors and placed in parallel. Each retaining unit includes the casing that covers the proximal end of the housing of each auto-injector from outside to cover the proximal end. The casing includes the distal opening into which the proximal end of the housing is inserted, the proximal wall abutting on or facing the proximal end face of the housing, and the cylindrical peripheral wall elastically deformed when the proximal end of the housing is housed in the casing to hold the proximal end of the housing housed in the casing by an elastic restoring force. Accordingly, the auto-injector retainer of the present invention appropriately bundles and retains a plurality of auto-injectors and appropriately assists simultaneous administration of a medical liquid by the plurality of auto-injectors without inhibiting functions of the auto-injectors.

In addition, the embodiment may be as follows.
(2) The auto-injector retainer according to (1), wherein the cylindrical peripheral wall has an inner surface provided with a rib protruding inward and abutting on an outer surface of the proximal end of the housing housed in the casing.
(3) The auto-injector retainer according to (2), wherein the rib included in the cylindrical peripheral wall is a rib extending in an axial direction or a circumferential direction of the cylindrical peripheral wall or a rib distributed on the inner surface of the cylindrical peripheral wall.
(4) The auto-injector retainer according to any one of (1) to (3), wherein the cylindrical peripheral wall includes a plurality of peripheral wall slits extending in an axial direction, and the peripheral wall slits extend from a proximal end of the cylindrical peripheral wall toward the distal opening and do not reach the distal opening.
(5) The auto-injector retainer according to (4), wherein the cylindrical peripheral wall is a polygonal cylindrical peripheral wall having a polygonal cross section perpendicular to the axial direction, and the peripheral wall slits are formed in a corner extending in the axial direction of the polygonal cylindrical peripheral wall.
(6) The auto-injector retainer according to (4) or (5), wherein the proximal wall includes a plurality of proximal wall slits continuous with the peripheral wall slits, the proximal wall being divided into a plurality of proximal segments by the plurality of proximal wall slits, and the proximal segments being displaceable along with elastic deformation of the cylindrical peripheral wall continuous with the proximal segments.
(7) The auto-injector retainer according to any one of (1) to (6), wherein the proximal wall includes an abutting projection protruding inward from a central portion of an inner surface of the casing on a proximal side, and the inner surface of the casing on the proximal side has a gap between a portion excluding the abutting projection and the proximal end face of the housing not to hinder elastic deformation of the cylindrical peripheral wall.
(8) The auto-injector retainer according to any one of (1) to (7), wherein the proximal end of the casing has an inner part provided with a protrusion protruding inward and abutting on the proximal end of the housing housed in the casing to press the proximal end of the housing housed in the casing toward the inner surface of the opposed cylindrical peripheral wall.
(9) The auto-injector retainer according to (8), including a proximal cutout formed by cutting out a part of the cylindrical peripheral wall facing the protrusion and a part of the proximal wall continuous to the cylindrical peripheral wall, and a portion of the cylindrical peripheral wall facing the protrusion where the proximal cutout is not formed serves as a clamping wall that clamps the proximal end of the housing together with the protrusion.
(10) The auto-injector retainer according to any one of (1) to (9), including a partition unit for maintaining the auto-injectors retained by two adjacent retaining units in a state of mutual separation.
(11) The auto-injector retainer according to (10), wherein the partition unit includes a casing partition that is a part of the casing in the two adjacent retaining units and has a predetermined thickness.
(12) The auto-injector retainer according to (10) or (11), wherein the partition unit includes an extending partition extending from distal ends of the retaining units, having a predetermined thickness, and placed between the auto-injectors retained by the two adjacent retaining units.

Furthermore, the drug administration device of the present invention is as follows.
(13) A drug administration device including: the auto-injector retainer according to any one of (1) to (12); and at least two of the auto-injectors.

The drug administration device includes the auto-injector retainer and at least two auto-injectors. Accordingly, the auto-injector retainer of the present invention appropriately bundles and retains a plurality of auto-injectors and appropriately assists simultaneous administration of a medical liquid by the plurality of auto-injectors without inhibiting functions of the auto-injectors.

1. An auto-injector retainer for use in simultaneous administration by a plurality of auto-injectors each having a housing that has a cylindrical shape and houses a prefilled syringe and a gasket pushing mechanism, the auto-injector retainer comprising:
a plurality of retaining units located in parallel, each retaining unit configured to retain a respective one of the auto-injectors; wherein:
each of the retaining units comprises a casing that covers a proximal end of the housing of the respective auto-injector; and
each casing comprises:
a distal opening into which the proximal end of the housing of the respective auto-injector is insertable,
a proximal wall configured to abut on or face a proximal end face of the housing of the respective auto-injector, and
a cylindrical peripheral wall configured to elastically deform when the proximal end of the housing of the respective auto-injector is housed in the casing and to hold the proximal end of the housing housed in the casing by an elastic restoring force.

2. The auto-injector retainer according to claim 1, wherein each casing comprises a rib protruding inward from an inner surface of the cylindrical peripheral wall, the rib configured to abut on an outer surface of the proximal end of the housing of the respective auto-injector housed in the casing.

3. The auto-injector retainer according to claim 2, wherein the rib of each casing extends in an axial direction or a circumferential direction of the cylindrical peripheral wall.

4. The auto-injector retainer according to claim 2, wherein each casing comprises a plurality of ribs protruding inward from an inner surface of the cylindrical peripheral wall, the rib configured to abut on an outer surface of the proximal end of the housing of the respective auto-injector housed in the casing.

5. The auto-injector retainer according to claim 1, wherein the cylindrical peripheral wall of each casing comprises a plurality of peripheral wall slits extending in an axial direction from a proximal end of the cylindrical peripheral wall toward the distal opening and without reaching the distal opening.

6. The auto-injector retainer according to claim 5, wherein the cylindrical peripheral wall of each casing is a polygonal cylindrical peripheral wall having a polygonal cross section perpendicular to the axial direction, and the peripheral wall slits are formed in a corner extending in the axial direction of the polygonal cylindrical peripheral wall.

7. The auto-injector retainer according to claim 5, wherein the proximal wall of each casing comprises a plurality of proximal wall slits continuous with the peripheral wall slits, the proximal wall being divided into a plurality of proximal segments by the plurality of proximal wall slits, and the proximal segments being displaceable along with elastic deformation of the cylindrical peripheral wall continuous with the proximal segments.

8. The auto-injector retainer according to claim 1, wherein the proximal wall of each casing comprises an abutting projection protruding inward from a central portion of an inner surface of the casing on a proximal side, and the casing is configured such that, when the respective housing is housed in the casing, a gap is located between the inner surface of the casing on the proximal side excluding the abutting projection and the proximal end face of the housing.

9. The auto-injector retainer according to claim 1, wherein a proximal end of each casing has an inner part comprising a protrusion protruding inward and configured to abut on the proximal end of the housing housed in the casing to press the proximal end of the housing housed in the casing toward an opposing portion of the inner surface of the cylindrical peripheral wall.

10. The auto-injector retainer according to claim 9, comprising:
in each casing, a proximal cutout that is cut out from a part of the cylindrical peripheral wall facing the protrusion and a part of the proximal wall continuous to the cylindrical peripheral wall; wherein:
a portion of the cylindrical peripheral wall facing the protrusion where the proximal cutout is not formed is configured to clamp the proximal end of the housing together with the protrusion.

11. The auto-injector retainer according to claim 1, comprising a partition configured to maintain the auto-injectors retained by two adjacent retaining units in a state of mutual separation.

12. The auto-injector retainer according to claim 11, wherein the partition comprises a casing partition that is a part of the casing in the two adjacent retaining units and has a predetermined thickness.

13. The auto-injector retainer according to claim 11, wherein the partition comprises an extending partition extending from distal ends of the retaining units, having a predetermined thickness, and located between the auto-injectors retained by the two adjacent retaining units.

14. A drug administration device comprising:
at least two auto injectors, each having a housing that has a cylindrical shape and houses a prefilled syringe and a gasket pushing mechanism; and
an auto-injector retainer for use in simultaneous administration by the at least two auto-injectors, the auto-injector retainer comprising:
a plurality of retaining units located in parallel, each retaining unit retaining a respective one of the auto-injectors, wherein:
each of the retaining units comprises a casing that covers a proximal end of the housing of the respective auto-injector, and
each casing comprises:
a distal opening into which the proximal end of the housing of the respective auto-injector is insertable,
a proximal wall abutting on or facing a proximal end face of the housing of the respective auto-injector, and
a cylindrical peripheral wall that is elastically deformed and holds the proximal end of the housing housed in the casing by an elastic restoring force.

15. A drug administration method comprising:
providing a drug administration device comprising:
at least two auto injectors, each having a housing that has a cylindrical shape and houses a prefilled syringe and a gasket pushing mechanism, and
an auto-injector retainer for use in simultaneous administration by the at least two auto-injectors, the auto-injector retainer comprising:
a plurality of retaining units located in parallel, each retaining unit retaining a respective one of the auto-injectors, wherein:
each of the retaining units comprises a casing that covers a proximal end of the housing of the respective auto-injector, and
each casing comprises:
a distal opening into which the proximal end of the housing of the respective auto-injector is insertable,
a proximal wall abutting on or facing a proximal end face of the housing of the respective auto-injector, and
a cylindrical peripheral wall that is elastically deformed and holds the proximal end of the housing housed in the casing by an elastic restoring force; and
while the at least two auto-injectors are held by the auto-injector retainer, simultaneously administering medical liquids from the at least two auto-injectors.
